(19) United States
(12) Patent Application Publication
(10) Pub. No.: US 2016/0324507 A1
(43) Pub. Date: Nov. 10, 2016

(54) SAMPLING UNIT, SAMPLING DEVICE AND METHOD FOR COLLECTING A SAMPLE

(71) Applicant: Moller Medical GmbH, Fulda (DE)

(72) Inventors: Bernhard Herget, Ehrenberg/Reulbach (DE); Andreas Schonberger, Fulda (DE); Christoph Traxler, Kunzell (DE)

(21) Appl. No.: 15/037,387

(22) PCT Filed: Nov. 18, 2013
(86) PCT No.: PCT/EP2013/003470

§ 371 Date: May 18, 2016

(51) Int. Cl. A61B 10/02

(52) U.S. Cl. CPC A61B 10/0266; A61B 2010/0208; A61B 2560/0443

(57) ABSTRACT

A sampling unit (1) with a needle, which forms a sample-holding space for receiving a sample and in or on which a cutting element (6) is mounted movably along the longitudinal axis, wherein the needle and the cutting element (6) can be moved relative to a stationary part by means of a driving device (2), is characterized in that the needle and the cutting element (6) are designed such that they can be moved from a first position into a second position by means of a driving element (9) of the driving device (2), wherein the needle is blocked in the second position, and the cutting element (6) can be moved further into a third position. A sampling device comprises, in addition to such a sampling unit (1), a driving device (2) interacting with the sampling unit (1).

The invention relates to a sampling unit, a sampling device comprising such a sampling unit, as well as a method for retrieving a sample from a material. In particular, the invention relates to a biopsy unit, a biopsy device with such a biopsy unit, as well as a method for retrieving a tissue sample.

Sampling devices are required for retrieving samples of animal, human, plant, and technical material or tissue, wherein, in particular, the retrieval of biopsy specimens in the course of a biopsy for evaluating potentially malignant tissue is paramount according to the invention.

The retrieval of a tissue sample may, for example, be performed by inserting a hollow needle having a relatively thin wall and comprising an inside mandrin (also called a “stylet”) in the direction of the tissue region of interest. The tip of the mandrin located inside the hollow needle is brought close to the tissue region of interest, whereby it is possible to observe the needle tip in the tissue using common examination methods, such as, for example, ultrasound. Subsequently, where applicable after or in combination with a retraction of the mandrin, the hollow needle is driven at high speed into the tissue section of interest—for example, by using a so-called firing apparatus—so that a tissue mass can be punched out of the surrounding connecting tissue using the hollow needle. By generating low pressure at the distal end of the hollow needle, the tissue sample is fixed in place in the hollow needle, whereby the punched-out tissue mass is torn from the connecting tissue as a result of the fixation when the hollow needle is retracted.

This avulsion functions satisfactorily only with relatively soft tissue. In particular, with firmer mixed tissue or with malignant tissue, it is, however, generally not possible to fix the tissue mass sufficiently by generating low pressure to be able to tear it from the connecting tissue.

In order to also allow for the retrieval of tissue samples from firmer and, in particular, malignant tissues, biopsy units were developed that comprise one or several lateral openings, into which surrounding tissue can enter after puncturing, i.e., after the quick driving forward of the needle. The tissue sample contained in the opening(s) is subsequently separated from the connecting tissue in a second step by means of a tubular cutting element that surrounds the needle and is provided with blades at the distal end. The tubular cutting element also serves to retain the tissue sample in the lateral opening(s), so that the tissue sample is prevented from slipping out when the biopsy unit is subsequently retracted. In order to also allow for the retrieval of tissue samples from firmer and, in particular, malignant tissues, biopsy units were developed that comprise one or several lateral openings, into which surrounding tissue can enter after puncturing, i.e., after the quick driving forward of the needle. The tissue sample contained in the opening(s) is subsequently separated from the connecting tissue in a second step by means of a tubular cutting element that surrounds the needle and is provided with blades at the distal end. The tubular cutting element also serves to retain the tissue sample in the lateral opening(s), so that the tissue sample is prevented from slipping out when the biopsy unit is subsequently retracted.

Such biopsy units have a disadvantageous ratio between the outer and inner diameters and thus between the tissue damage caused during puncturing and the volume of the tissue sample.

Moreover, appropriate two-stage firing devices are used for such biopsy units on account of the two-stage driving forward of first the needle and then the tubular cutting element, these two-stage firing devices comprising two independent driving mechanisms, which must generally be activated separately. These firing devices are not only structurally complex, but also comparatively difficult to use.

DE 10 2007 002 855 A1 discloses a biopsy unit which is distinguished by an improved ratio between tissue damage caused and the volume of the tissue sample. For this purpose, the biopsy unit comprises, like the units that are based upon severing the tissue sample from the connecting tissue by generating low pressure, a hollow needle with a (first) hollow space, which is open on both sides and which contains the mandrin. Furthermore, the hollow needle forms a second hollow space, which also extends in the longitudinal direction of the hollow needle and in which a resiliently flexible cutting element is movably mounted. The cutting element is used to sever the tissue sample received in the first hollow space from the rest of the tissue and to subsequently close the proximal opening of the first hollow space of the hollow needle so that the sample tissue is safely prevented from slipping out of the first hollow space when the hollow needle is retracted. For this purpose, the cutting element is driven forward at high speed after the puncturing of the tissue of interest by means of the hollow needle, whereby the front end of the cutting element forming a blade is deflected during the movement diagonally with respect to the longitudinal axis of the hollow needle, whereby the tissue sample received in the first hollow space of the hollow needle is separated from the connecting tissue located in front of the hollow needle, and the proximal opening of the first hollow space of the hollow needle is closed simultaneously.

Since the function of the biopsy units known from DE 10 2007 002 855 A1 is also based upon a two-stage driving forward of the hollow needle and cutting element, it can be used with an appropriate design with the same firing devices that are also used for biopsy units with lateral sample-holding recesses. This may be advantageous, since the same firing devices can be used for different biopsy units, but does not change the basically high complexity of the firing devices with respect to their structural design and their operation.

Starting from this prior art, the invention was based upon the task of providing a biopsy unit that allows for easy use and/or simple structural design of a driving device used or combined with this biopsy unit.

This aim is achieved by a sampling unit according to claim 1. A sampling device comprising such a sampling unit is the object of claim 12. A method that can be performed using such a sampling device is the object of claim 16. Advantageous embodiments of the sampling unit according to the invention and of the sampling device according to the invention are the object of additional claims and result from the following description of the invention.

The invention is based upon the idea that a simple application of a biopsy unit, as well as a structurally simple design of a driving device used with the biopsy unit, can be achieved if the two-stage design of the drive, i.e., the temporal delay between the driving of the tip of the hollow needle and the tip of the cutting element, does not have to be brought about by sequentially activating two driving mechanisms, but instead by an appropriate functional interaction of the elements of the sampling unit itself—in particular, of the (hollow) needle—as well as the cutting element or elements connected thereto. In this respect, this idea is based upon the realization that a temporal delay during the penetration of the connecting tissue by first the hollow needle and then the cutting element does not necessarily have to be associated with a temporal delay in the driving of these elements. Rather, the temporal delay during penetration can also be achieved if both elements are at times driven forward simultaneously and, in particular, in combination with each other, where one of these elements, namely, the (hollow) needle, starts this simultaneous movement with a (temporal or spatial) “head start.” The driving of the (hollow) needle is thus already ended, while the cutting element is still completing its movement.

Such a sampling unit allows for a sample retrieval that, functionally, occurs in accordance with the prior art, wherein, however, only one (single) driving mechanism can be provided for the driving of the (hollow) needle and the cutting element.

Accordingly, a sampling unit according to the invention—in particular, one that is used as a biopsy unit—comprises at least one needle, which forms at least one sample-holding space for receiving a sample, wherein a cutting element is mounted movably along the longitudinal axis in or on the needle. The needle and the cutting element are designed such that they are slidable (in particular, using a driving device) relative to the stationary part of the sampling unit. Here, the needle and the cutting element are designed such that they are slidable (in particular, using a driving element of the driving device) from a first position into a second position, wherein the needle is blocked in the second position and thus cannot be moved further, while the cutting element can be moved further into a third position. The relative movement between the cutting element and the needle during the movement of the cutting element from the second position into the third position results in the cutting element preferably completely separating the material (tissue) of the sample located in the sample-holding space from the surrounding material (tissue) and preferably, also, in the sample-holding space being (preferably completely) closed off toward the outside by means of the cutting element, in order to prevent a loss of the sample from the sample-holding space.

In the process, the first and second positions of the hollow needle and the cutting element may be defined individually. The second position of the hollow needle may, in particular, be the end position of its driving movement, whereas the second position of the cutting element is an interim position. In particular, the third position may be the end position of the driving movement of the cutting element.

A sampling device according to the invention, which can be used, in particular, as a biopsy device, comprises, in addition to the sampling unit according to the invention, at least one driving device comprising the driving element and interacting with the sampling unit.

Here, the sampling unit can be mounted interchangeably in or on the driving device. Among other things, this allows for providing the sampling unit for one-time use and the driving device for multiple uses. In this way, the costs for the individual sample retrievals can be kept low. It is, however, also possible to provide for the driving device to be for only one-time use as well. In this case, it may also be advantageous not to provide an interchangeable mounting of the sampling unit in or on the driving device.

A design of the entire sampling device for one-time use may be economical, in particular, due to the simple design of the driving device, for which a design with only one single driving mechanism may be sufficient due to the design of the sampling unit according to the invention.

Using such a sampling device, a method for retrieving a sample from a material can be performed, in which the needle and the cutting element are driven forward together from a first into a second position using the driving element of the driving device, whereby the sample is received in the sample-holding space of the needle, and the movement of the needle into the second position is stopped, whereas the cutting element is moved further to a third position using the driving element, whereby the sample is (preferably completely) separated from the material located outside the sample-holding space.

Even though the use of only one driving mechanism is sufficient for using the sampling unit according to the invention, the sampling unit can advantageously also be designed such that it can be used with driving devices that are provided for use with conventional sampling units and therefore comprise more than one—in particular, two—driving mechanisms. For this purpose, it can be provided that only one of the two or several driving mechanisms is employed for the use of a sampling unit according to the invention. In this way, it can be achieved that already existing driving devices can continue to be used when transitioning from conventional sampling units to sampling units according to the invention.

The sampling unit according to the invention can be designed in the manner described with one or several lateral hollow spaces, wherein the cutting element having a tubular shape (which is not restricted to a circular cross section, but, rather, can be designed arbitrarily) is movably mounted on the needle (which may also have any cross-sectional shape).

Preferably, it is, however, provided that the sampling unit is designed with a hollow needle, which forms a distal opening that allows distal access to a (first) hollow space of the hollow needle used to receive the sample. In a preferred embodiment of the sampling unit, it can then also be provided that in this (first) hollow space of the hollow needle, a mandrin is mounted movably along the longitudinal axis. This mandrin can preferably be used to prevent material—in particular, tissue—from entering the (first) hollow space, unless this is provided for—thus, in particular, when the distal end of the hollow needle is moved through material (tissue) toward a material or tissue section of interest. In order to simplify such a movement, the distal end of the mandrin can also be designed to be tapered. By driving the hollow needle using the driving device from the first into the second position, the hollow needle can subsequently be moved on the mandrin so far that space is cleared for receiving the sample in the (first) hollow space and a sample-holding space thus created.

The “distal” end of the sampling unit is the end that is driven into the material in order to retrieve the sample. The proximal end is the respective other end of the sampling unit, which is generally arranged in or on the driving device.

The static element can preferably be the mandrin or an element firmly connected to the mandrin (at least with respect to the direction of movement of the hollow needle and the cutting element). The sampling device according to the invention can then also preferably comprise fixation means for directly or indirectly (in particular, via an element firmly connected to the mandrin) fixing the mandrin to a housing of the driving device. These fixation means can preferably be designed as plug connections (preferably with a plug-in direction that is orthogonal to the direction of movement of the hollow needle and the cutting element).

In another preferred embodiment of the sampling unit according to the invention, it can also be provided that the cutting element is mounted movably along the longitudinal axis in a hollow space of the needle designed as a hollow needle. A secure guiding of the relative movement between the hollow needle and the cutting element (at least during its movement from the second into the third position) can thereby be achieved. In addition, the cutting element is thereby securely received in the hollow needle, so that the danger of damage during handling of the sampling device can be reduced. The hollow space is preferably a (second) hollow space other than the (first) hollow space provided for receiving the sample and, where applicable, a mandrin.

The blocking of the needle in its second position can preferably be achieved by the needle or an element firmly connected thereto (at least with respect to the direction of movement) hitting a stop member. This stop member can be part of the sampling unit and/or the driving device.

In a preferred embodiment of the sampling unit according to the invention, a locking element may be provided that couples the needle with the cutting element in a locked position and thus secures a defined relative position of these elements to one another. It can thereby be achieved that the needle and the cutting element are moved together from the first position in the direction of the second position, preferably using the same driving element. Furthermore, means for unlocking the locking element can then be provided, whereby the coupling between the needle and the cutting element can be disconnected. This allows for the cutting element to be moved independently of the needle to the third position.

Preferably, for this purpose, it can be provided that the locking element is moved as well, when the needle and the cutting element are moved from the first position in the direction of the second position and, in particular (at least), until the second position is reached, where the locking element is guided in a control groove. The locking element can then also preferably be moved from the locked position by means of the control groove before or upon reaching the second position (which may be an individual position for the locking element). For this purpose, the locking element can be moved in a lateral direction and, in particular, orthogonally to the driving direction of the needle and the cutting element.

In another preferred embodiment of the sampling unit according to the invention, a removable securing element can be provided, which fixes the needle in the second position and the cutting element in the second or third position. The securing element can, in particular, be used as a transportation safety device so that the fixation of the position of the needle, cutting element, and preferably also the mandrin relative to each other can also be realized when the sampling unit is not yet mounted in or on the driving device.

Preferably, it can also be provided that the securing element of a sampling unit mounted in a housing of the driving device protrudes from the housing of the driving device outward. In such an embodiment, the securing element can, in particular, be used as a handle, which facilitates the insertion and removal of the sampling unit from the housing of the driving device. In order to be suitable as a handle, the securing element can, in particular, form a sufficiently large gripping surface.

Accordingly, in a preferred embodiment of the sampling device according to the invention, it can also be provided that the sampling unit can be inserted into or attached to the driving device by means of the position —which has been secured by the securing element—of the needle, cutting element, and, where applicable, the mandrin (relative to each other). Preferably, in particular, the driving element, which interacts directly or indirectly with the needle and the cutting element, and thus the entire driving device, can then be located in a neutral position, which it also assumes after the sample has been retrieved, i.e., after the complete driving forward of both the needle and the cutting element. In a spring-based driving device, in which the force required to drive the driving element, and thus the needle and the cutting element, is generated by at least one, and preferably one single, pretensioned spring element, the sampling unit can thus be inserted or attached, as well as removed, from the driving device after retrieval of the sample when the spring element is released or only slightly pretensioned. In this way, the handling safety for the sampling device can be increased.

In order to then move the needle and the cutting element into the first position, which can be an initial position of the sampling device for the retrieval of the sample, it can be provided that the needle and the cutting element are designed such that they are slidable from the third and second position into the first position by means of a (preferably single) retracting element of the driving device. This movement of the retracting element can, in particular, be connected to a (further) pretensioning of the spring element of a spring-based driving device of the sampling device.

The retracting element may be a different component than the driving element. The driving element can, however, also be so designed and arranged that it also functions as a retracting element.

In a preferred embodiment of the sampling unit according to the invention, additional means can be provided which temporarily fix the needle, while the cutting element is moved from the third into the second position. In this way, a forward displacement (of the distal end) of the needle relative to the (distal end of the) cutting element(s) can easily be realized. By means of this forward displacement, which can be maintained until the first position is reached, it can be achieved that the driving movement of the needle is stopped (in the second position), before the driving movement of the cutting element is stopped (in the third position). In the process, the stopping of the driving movements of both elements can also be carried out by means of the same stop member.

The means for the temporary fixation can be designed, for example, in a structurally simple manner as a snap connection, which is, in particular, disconnected if a defined limit force is exceeded.

In another preferred embodiment of the sampling unit according to the invention, means for removably fixing the cutting element in the third position can be provided. This can, in particular, be used to safely fix the cutting element in the third position, in which it preferably closes the sample-holding space and thus encloses a sample in the needle, while the sampling unit is retracted or otherwise handled. Owing to the detachability of the fixation of the cutting element in the third position, the sample can be removed as needed. Preferably, these fixation means can also be designed in a structurally simple manner as snap connections.

Preferably, in the sampling unit according to the invention, it can be provided that all elements be made of plastic, with the exception, if necessary, of the needle, the cutting element, and/or the mandrin, which may, in particular, be formed of a metal. In doing so, a production of the plastic elements as injection molded parts can be advantageous—in particular, in a large-series production of the sampling unit.

The invention is described in more detail below with reference to an exemplary embodiment illustrated in the figures. The figures show:

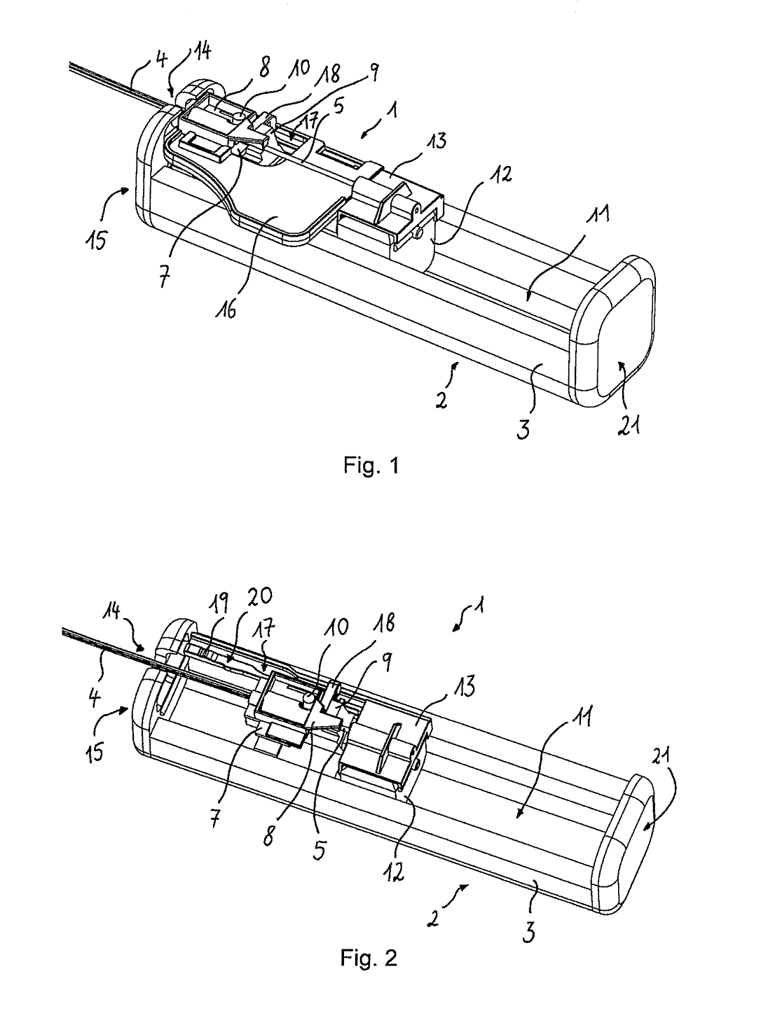

FIG. 1: a sampling device according to the invention in a neutral position prior to the retrieval of a sample;

FIG. 2: the sampling device in an initial position prior to the retrieval of a sample;

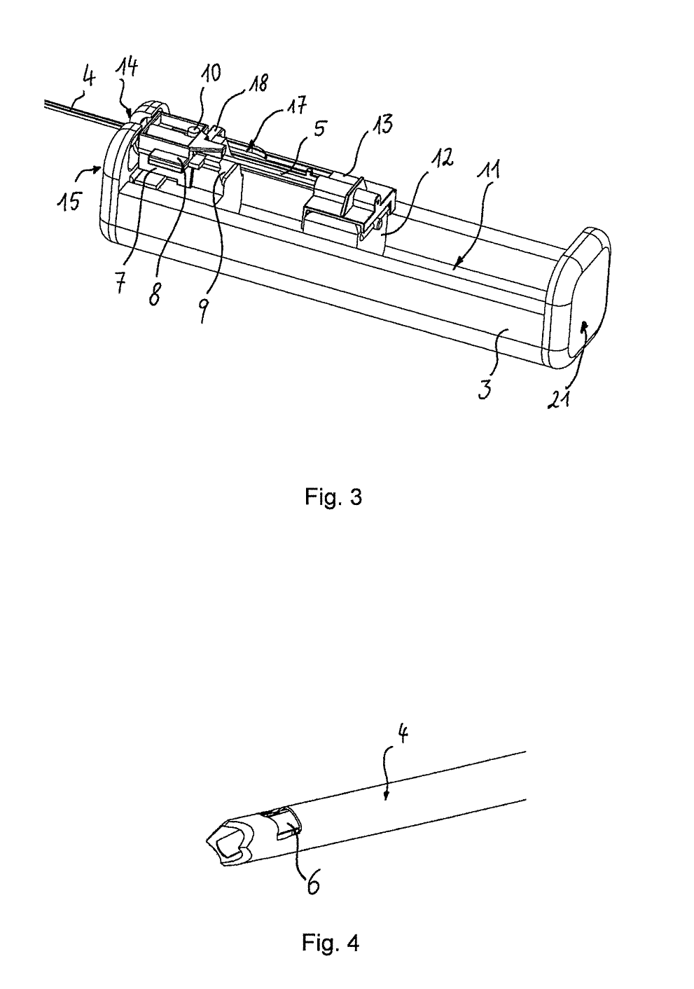

FIG. 3: the sampling device in a neutral position after the retrieval of a sample; and

FIG. 4: a detailed view of the distal end of the sampling unit of the sampling device according to FIG. 3.

FIGS. 1 through 4 show a sampling device according to the invention, which is, in particular, provided as a biopsy device and thus for the retrieval of a tissue sample from a human, animal, or plant body.

The sampling device comprises a sampling unit 1, as well as a driving device 2, which is designed as a so-called firing device. Here, a one-time use of the sampling unit 1 is provided for, while the driving device 2 can be used multiple times, i.e., successively, with a plurality of sampling units 1. For this purpose, the sampling unit 1 is interchangeably mounted in a housing 3 of the driving device 2.

The sampling unit 1 comprises a hollow needle 4, which forms two hollow spaces (not shown), which extend in the direction of the longitudinal axis and are open and thus accessible at both ends of the hollow needle 4. A first of the hollow spaces is used to accommodate a mandrin 5 of the sampling unit 1, as well as to receive a sample after an appropriate relative movement of the mandrin 5 to the hollow needle 4. In the second of the hollow spaces, a stripe-shaped, resiliently elastic cutting element 6 is mounted movably in the longitudinal direction, whose distal end 6 forms a blade (cf. FIG. 4). When the cutting element 6 is moved in the second hollow space in the direction of the distal end of the sampling unit 1, the distal end of the cutting element 6 is deflected in the lateral direction and thereby deformed into an arc shape, whereby it passes over the distal opening of the first hollow space of the hollow needle 4 (cf. FIG. 4). In this way, a (tissue) sample, which is arranged inside the sample-holding space formed by the first hollow space of the hollow needle 4, can be severed from material (tissue) located in front of the distal end of the hollow needle 4. At the same time, the cutting element 6 closes the distal opening of the first hollow space and thus safely encloses the sample in the sample-holding space.

The distal end of the hollow needle 4 and the distal end of the cutting element 6 are each firmly connected to an adapter element 7, 8. A driving element 9 as well as a retracting element 10 of the driving device 2 can engage with these adapter elements 7, 8 in order to effect the movements of the hollow needle 4 and of the cutting element 6 provided for the retrieval of the sample.

The oblong driving device 2 comprises a base part, which is shown in the figures and which forms a recess 11 extending in the direction of the longitudinal axis, as well as a cover, which is not shown in the figures and by means of which the base part can be closed. In a central section of the recess 11, a stationary wall element 12 of the driving device 2 is arranged. The upper edge of the wall element 12 engages with the retaining element 13 of the sampling unit 1 and thus fixes it, when the sampling unit 1 is inserted into the driving device 2. This retaining element 13, as well as the mandrin 5 firmly connected thereto, are thus stationarily arranged with respect to the housing 3 of the driving device 2.

In the section of the recess 11 that extends from the wall element 12 to the front wall 15 of the housing 3, which forms a passage opening 14 for the sampling unit 1, a slide of a driving mechanism of the driving device 2 is mounted movably in the direction of the longitudinal axis. A spring element (not shown) is supported between the slide and the wall element 12. Here, the spring element is not or only slightly pretensioned in the neutral position of the sampling device shown in FIGS. 1 and 3. In this neutral position, the slide is also moved as far as possible in the direction of the front wall 15 comprising the passage opening 14.

In this neutral position, the sampling unit 1 can be inserted into the driving device 2, whereby the position of the elements of the sampling unit 1—i.e., in particular, the hollow needle 4, the cutting element 6, and the mandrin 5, including the retaining element 13 connected thereto—is fixed by means of a securing element 16. In the process, the securing element 16 forms a handle area, which simplifies handling of the sampling unit 1 and also protrudes for this purpose in particular from the exterior of the housing 3 of the driving device 2.

The slide forms the driving element 9, as well as the retracting element 10, of the driving device 2. By means of the driving element 9, a force generated by the pretensioned spring element can be transferred to the adapter elements 7, 8 connected to the hollow needle 4 and the cutting element 6.

Here, the pin-shaped retracting element 10 extends through the passage openings of the adapter elements 7, 8, wherein the passage opening (not shown) of the adapter element 7 connected to the hollow needle 4 is designed as an oblong hole extending in the direction of the longitudinal axis of the hollow needle 4, whereby a relative movement between the retracting element 10 and the hollow needle 4 is possible within defined limits.

If a new sampling unit 1 has been inserted into the driving device 2 (cf. FIG. 1), the hollow needle 4 is located in a second position, and the cutting element 6 is located in a third position. After the securing element 16 has been removed, the driving mechanism of the driving device 2 can be tensioned. This is carried out by means of a tension lever (not shown in the figures), through which the slide is moved in the direction of the wall element 12 while further pretensioning the spring element. In the process, the retracting element 10 directly takes along the cutting element 6, while a relative movement of the retracting element 10 guided in the oblong hole with respect to the hollow needle 4 occurs first. The taking along of the hollow needle 4 does not occur until the retracting element 10 is stopped by the end of the oblong hole facing the wall element 12. At this point in time, the cutting element 6 is already located in its second position. In order to avoid an undesirable co-movement of the hollow needle 4 due to friction, a snap connection is formed between the adapter element 7 connected to the hollow needle 4 and an element which forms a control groove 17 for a locking element 18 and which is designed as one piece with the retaining element 13 for the mandrin. For this purpose, this element comprises a snap recess 19 with which a snap projection (not shown) of the adapter 7 engages. In order to disconnect the snap connection, a defined breakaway force is required, which is exceeded when the retracting element 10 is stopped by the end of the oblong hole that is located closer to the wall element 12.

From then on, a combined movement of the hollow needle 4 and the cutting element 6 occurs, wherein an offset is formed between the adapter elements 7, 8. This offset constitutes a gap between the adapter 7 connected to the hollow needle 4 and the driving element 9 of the slide. During the movement from the second position in the direction of the first position shown in FIG. 2, a locking element 18, which is guided in the control groove 17 and taken along, is automatically slid into this gap in an orthogonal movement. To this end, the control groove 17 has in one section an s-shaped run, by means of which the control groove 17 is divided into two sections, where the section that is located closer to the wall element 12 runs parallel to the longitudinal axis of the hollow needle 4 at a smaller distance than the other section.

In the initial position shown in FIG. 2 of the sampling device, both the hollow needle 4 and the cutting element 6 are in their first positions. The spring element is significantly pretensioned, and the slide is secured in its position by means of a retaining device (not shown). By releasing the retaining device, the sampling device can be “fired,” whereby the driving element 9 of the slide suddenly moves the hollow needle 4 together with the cutting element 6 from the first position in the direction of the second position due to the spring loading. In the process, the driving element 9 comes into direct contact with the adapter 8 connected to the cutting element 6, while the locking element 18 is initially still arranged between the driving element 9 and the adapter 7 connected to the hollow needle 4 and thus couples the movement of the hollow needle 4 with the movement of the cutting element. As a result of the s-shaped run of the control groove 17, the locking element 18 is not guided laterally from the gap formed between the adapter 7 and the driving element 9 until shortly before the (respective) second position is reached. This results in the driving element 9 still being able to continue to move, when the adapter element 7 connected to the hollow needle 4 is stopped in the second position by the front wall 15 of the housing 3 comprising the passage opening 14. The driving element 9 thereby moves the adapter 8 connected to the cutting element 6 even further until it, too, is stopped in the third position of the cutting element 6 by the front wall 15 of the housing 3. In doing so, a movement of the cutting element 6 in the second hollow space of the hollow needle 4 and the already described (preferably complete) severing of the material (tissue), as well as the closing of the distal opening of the first hollow space of the hollow needle 4, occur.

FIG. 3 shows the positions of the hollow needle 4 and the cutting element 6, along with the adapter elements 7, 8, after the retrieval of a sample. Here, the hollow needle 4 is in the second position, and the cutting element 6 is in the third position. These positions correspond to the positions according to FIG. 1. It is thus possible to insert the securing element 16 into the rest of the sampling unit 1 again and to remove the latter from the driving device 2.

Generally, the insertion of the securing element 16, for which a removal of the cover may be required, does not occur until after the sampling unit 1 has been retracted from the material (tissue) from which the sample was retrieved. In order to avoid an undesirable slide of the cutting element 6 in the second hollow space of the hollow needle 4 during the retraction of the sampling unit 1, which would be associated with a (partial) release of the enclosed sample, the element forming the control groove 17 forms a snap projection 20. This projection holds the adapter 8 connected to the cutting element 6 in the third position, unless a defined breakaway force is exerted on this projection in the direction of the wall element 12 (as occurs during tensioning of the driving mechanism).

The driving device 2 is a driving device that is also suitable for use with a conventional sampling unit, in which a two-stage movement of the hollow needle and the cutting element is realized by means of two separate driving mechanisms. For the sampling unit 1 according to the invention, only one driving mechanism—namely, the one located closer to the front wall 15—is used. The second driving mechanism (not shown here) arranged in the section of the recess 12 that runs between the wall element 12 and the other front wall 21, however, remains unused.

### LIST OF REFERENCE SYMBOLS

1. Sampling unit
2. Driving device
3. Housing
4. Hollow needle
5. Mandrin
6. Cutting element
7. Adapter
8. Adapter
9. Driving element
10. Retracting element
11. Recess
12. Wall element
13. Retaining element
14. Passage opening
15. Front wall
16. Securing element
17. Control groove
18. Locking element
19. Snap recess
20. Snap projection
21. Front wall

1-16. (canceled)

17. A sampling unit, comprising:
a stationary part;
a needle having a sample-holding space for receiving a sample, the needle having a longitudinal axis;
a cutting element disposed in or on the needle and mounted movably along the longitudinal axis;
wherein the needle and the cutting element are movable relative to the stationary part;
the needle and the cutting element being slidable from a first position into a second position, wherein the needle is blocked in the second position, and the cutting element is further movable into a third position.

18. The sampling unit according to claim 17, wherein the needle is a hollow needle having a hollow space therein, the sampling unit further comprising a mandrin mounted movably along the longitudinal axis in the hollow space of the hollow needle.

19. The sampling unit according to claim 17, wherein the needle is a hollow needle having a hollow space therein, the cutting element being mounted movably along the longitudinal axis in the hollow space of the hollow needle.

20. The sampling unit according to claim 17, further comprising:
a locking element having a selectable locked position, the locking element when in the locked position coupling the needle with the cutting element; and
means for unlocking the locking element.

21. The sampling unit according to claim 20, wherein the locking element is moved with the movement of the needle and the cutting element from the first position into the second position.

22. The sampling unit according to claim 21, further comprising a control groove, wherein the locking element is guided in the control groove, the locking element being moved out of the locked position by the control groove before or when the second position is reached.

23. The sampling unit according to claim 17, further comprising a removable securing element operable to fix the needle in the second position and the cutting element in the second or third position.

24. The sampling unit according to claim 17, further comprising means for temporarily fixing the needle, while the cutting element is movable from the third into the second position.

25. The sampling unit according to claim 24, wherein the means for temporarily fixing the needle comprises a snap connection.

26. The sampling unit according to claim 17, further comprising means for removably fixing the cutting element in the third position.

27. The sampling unit according to claim 26, wherein the means for removably fixing the cutting element comprises a snap connection.

28. A sampling device, comprising:
a sampling unit according to claim 17; and
a driving device interacting with the sampling unit for moving the needle and the cutting element.

29. The sampling device according to claim 28, wherein the driving device has a housing, the sampling device further comprising a fixation means for fixing the stationary part of the sampling unit to the housing of the driving device.

30. The sampling device according to claim 28, wherein the sampling unit is mounted interchangeably in or on the driving device.

31. The sampling device according to claim 30, wherein the driving device has a housing, the sampling device further comprising a fixation means for fixing the stationary part of the sampling unit to the housing of the driving device.

32. The sampling device according to claim 28, wherein the sampling unit further comprises a removable securing element operable to fix the needle in the second position and the cutting element in the second or third position, the driving device has a housing, and the securing element protrudes outwardly from the housing of the driving device.

33. A method for retrieving a sample from a material, comprising:
driving a needle forming a sample-holding space forward into the material in order to receive a sample in a sample-holding space of the needle, the needle having a longitudinal axis;
driving forward a cutting element, which is mounted movably along the longitudinal axis in or on the needle, so as to separate the sample from the material located outside the sample-holding space;
wherein the needle and the cutting element are driven forward together from a first into a second position by means of a driving element of a driving device, whereby the sample is received in the sample-holding space; and
wherein the movement of the needle is stopped in the second position, whereas the cutting element is moved further to a third position by means of the driving element, whereby the sample is separated from the material located outside the sample-holding space.
